SEQUENCE LISTING (27 sequences)

SEQ ID NO: 1 - protein, 10 aa, synthetic construct
  REGION 1..10: note = HCDR1 of Anti-BCMA VH
GFTFSNFGMH

SEQ ID NO: 2 - protein, 9 aa, synthetic construct
  REGION 1..9: note = HCDR2 of Anti-BCMA VH
VIWSDETNR

SEQ ID NO: 3 - protein, 16 aa, synthetic construct
  REGION 1..16: note = HCDR3 of Anti-BCMA VH
DQQYCSSDSCFTWFDP

SEQ ID NO: 4 - protein, 15 aa, synthetic construct
  REGION 1..15: note = LCDR1 of Anti-BCMA VL_v2, of Anti-BCMA VL_v6 and of Anti-BCMA VL_v7
CASSTGTVTPSNYAN

SEQ ID NO: 5 - protein, 15 aa, synthetic construct
  REGION 1..15: note = LCDR1 of Anti-BCMA VL_parent and of Anti-BCMA VL_v1
CRSSTGTVTPSNYAN

SEQ ID NO: 6 - protein, 15 aa, synthetic construct
  REGION 1..15: note = LCDR1 of Anti-BCMA VL_v3
CASSTGAVTPSNYAN

SEQ ID NO: 7 - protein, 15 aa, synthetic construct
  REGION 1..15: note = LCDR1 of Anti-BCMA VL_v4
CASSTGAVTPGYYAN

SEQ ID NO: 8 - protein, 15 aa, synthetic construct
  REGION 1..15: note = LCDR1 of Anti-BCMA VL_v5
CASSTGAVTSGYYAN

SEQ ID NO: 9 - protein, 7 aa, synthetic construct
  REGION 1..7: note = LCDR2 of Anti-BCMA VL_parent, of Anti-BCMA VL_v1, of Anti-BCMA VL_v2, of Anti-BCMA VL_v6 and of Anti-BCMA VL_v7
DNNSRPP

SEQ ID NO: 10 - protein, 7 aa, synthetic construct
  REGION 1..7: note = LCDR2 of Anti-BCMA VL_v3
DNNIKPP

SEQ ID NO: 11 - protein, 7 aa, synthetic construct
  REGION 1..7: note = LCDR2 of Anti-BCMA VL_v4
DNNNKPP

SEQ ID NO: 12 - protein, 7 aa, synthetic construct
  REGION 1..7: note = LCDR2 of Anti-BCMA VL_v5
DNNIKPS

SEQ ID NO: 13 - protein, 9 aa, synthetic construct
  REGION 1..9: note = LCDR3 of Anti-BCMA VL_parent, of Anti-BCMA VL_v1, of Anti-BCMA VL_v2, of Anti-BCMA VL_v6 and of Anti-BCMA VL_v7
ALWFGNQWV

SEQ ID NO: 14 - protein, 9 aa, synthetic construct
  REGION 1..9: note = LCDR3 of Anti-BCMA VL_V3
ALWYGGQWV

SEQ ID NO: 15 - protein, 9 aa, synthetic construct
  REGION 1..9: note = LCDR3 of Anti-BCMA VL_v4 and of Anti-BCMA VL_v5
ALYYGGQWV

SEQ ID NO: 16 - protein, 125 aa, synthetic construct
  REGION 1..125: note = Anti-BCMA VH
QVQLVESGGGVVQPGRSLRLSCAASGFTFSNFGMHWVRQAPGRGLEWVAVIWSDETNRYYADSVKGRFTVSRDNVKSTVYLQMNSLISEDTAVYYCARDQQYCSSDSCFTWFDPWGQGTLVTVSS

SEQ ID NO: 17 - protein, 109 aa, synthetic construct
  REGION 1..109: note = Anti-BCMA VL_parent
QVVVTQESALTTSPGETVTLTCRSSTGTVTPSNYANWVQEKPDHFFTGLIGDNNSRPPGVPARFSASLIGDKAALTITGAQTEDEAMYFCALWFGNQWVFGGGTKVTVL

SEQ ID NO: 18 - protein, 109 aa, synthetic construct
  REGION 1..109: note = Anti-BCMA VL_v1
QTVVTQEPSLTVSPGGTVTLTCRSSTGTVTPSNYANWVQQKPDHFFTGLIGDNNSRPPGVPARFSASLIGDKAALTLSGVQPEDEAEYYCALWFGNQWVFGGGTKLTVL

SEQ ID NO: 19 - protein, 109 aa, synthetic construct
  REGION 1..109: note = Anti-BCMA VL_v2
QTVVTQEPSLTVSPGGTVTLTCASSTGTVTPSNYANWVQQKPGQAPRGLIGDNNSRPPGTPARFSASLLGGKAALTLSGVQPEDEAEYYCALWFGNQWVFGGGTKLTVL

SEQ ID NO: 20 - protein, 109 aa, synthetic construct
  REGION 1..109: note = Anti-BCMA VL_v3
QTVVTQEPSLTVSPGGTVTLTCASSTGAVTPSNYANWVQQKPGQAPRGLIGDNNIKPPWTPARFSGSLLGGKAALTLSGVQPEDEAEYYCALWYGGQWVFGGGTKLTVL

SEQ ID NO: 21 - protein, 109 aa, synthetic construct
  REGION 1..109: note = Anti-BCMA VL_v4
QTVVTQEPSLTVSPGGTVTLTCASSTGAVTPGYYANWVQQKPGQAPRGLIGDNNNKPPWTPARFSGSLLGGKAALTLSGVQPEDEAEYYCALYYGGQWVFGGGTKLTVL

SEQ ID NO: 22 - protein, 109 aa, synthetic construct
  REGION 1..109: note = Anti-BCMA VL_v5
QTVVTQEPSLTVSPGGTVTLTCASSTGAVTSGYYANWVQQKPGQAPRALIGDNNIKPSWTPARFSGSLLGGKAALTLSGVQPEDEAEYYCALYYGGQWVFGGGTKLTVL

SEQ ID NO: 23 - protein, 109 aa, synthetic construct
  REGION 1..109: note = Anti-BCMA VL_v6
QTVVTQEPSLTVSPGGTVTLTCASSTGTVTPSNYANWVQQKPGQFPRGLIGDNNSRPPGTPARFSASLLGGKAALTLSGVQPEDEAEYYCALWFGNQWVFGGGTKLTVL

SEQ ID NO: 24 - protein, 109 aa, synthetic construct
  REGION 1..109: note = Anti-BCMA VL_v7
QTVVTQEPSLTVSPGGTVTLTCASSTGTVTPSNYANWVQQKPGQAFRGLIGDNNSRPPGTPARFSASLLGGKAALTLSGVQPEDEAEYYCALWFGNQWVFGGGTKLTVL

SEQ ID NO: 25 - protein, 15 aa, synthetic construct
  REGION 1..15: note = CDR-L1 of anti-BCMA
  VARIANT 2: note = X in position 2 is R or A
  VARIANT 7: note = X in position 7 is T or A
  VARIANT 11: note = X in position 11 is S or G
  VARIANT 12: note = X in position 12 is N or Y
CXSSTGXVTPXXYAN

SEQ ID NO: 26 - protein, 7 aa, synthetic construct
  REGION 1..7: note = CDR-L2 of anti-BCMA
  VARIANT 4: note = X in position 4 is S, I, or N
  VARIANT 5: note = X in position 5 is R or K
DNNXXPP

SEQ ID NO: 27 - protein, 9 aa, synthetic construct
  REGION 1..9: note = CDR-L3 of anti-BCMA
  VARIANT 3: note = X in position 3 is W or Y
  VARIANT 4: note = X in position 4 is F or Y
  VARIANT 6: note = X in position 6 is N or G
ALXXGXQWV

(19) United States
(12) Patent Application Publication
(10) Pub. No.: US 2024/0117062 A1
(43) Pub. Date: Apr. 11, 2024

(54) ANTI-BCMA ANTIBODIES

(71) Applicant: SANOFI, Paris (FR)

(72) Inventors: Jochen BENINGA, Frankfurt am Main (DE); Alessandro MASIERO, Paris (FR); Sambasiva RAO, Cambridge, MA (US); Leila SEVIGNY, Cambridge, MA (US); Yanfeng ZHOU, Cambridge, MA (US)

(21) Appl. No.: 18/324,668

(22) Filed: May 26, 2023

(30) Foreign Application Priority Data
May 27, 2022 (EP) 22305784.5

(51) Int. Cl. C07K 16/28

(52) U.S. Cl. CPC C07K 16/2878; C07K 2317/21; C07K 2317/24; C07K 2317/31

(57) ABSTRACT

Antibodies and antigen-binding fragments thereof that bind to B-cell maturation antigen (BCMA), and methods of using the same, are provided.

### RELATED APPLICATIONS

This application claims priority to European Patent Application No. 22305784.5, filed May 27, 2022, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on May 26, 2023, is named 740012_SA9-335_ST26.xml and is 39,280 bytes in size.

### FIELD OF THE INVENTION

This disclosure relates to novel antibodies and antigen-binding fragments thereof that bind to B-cell maturation antigen (BCMA), and methods of using the same.

### BACKGROUND

B-cell maturation antigen (BCMA) is a member of the tumor necrosis receptor (TNFR) family and is expressed on cells of the B-cell lineage. BCMA expression is the highest on terminally differentiated B cells and is involved in mediating the survival of plasma cells for maintaining long-term humoral immunity. The expression of BCMA has been linked to a number of cancers and plasma cell malignancies, such as multiple myeloma (MM). RNA has been detected universally in multiple myeloma cells, and BCMA protein has been detected on the surface of plasma cells from multiple myeloma patients by several investigators. As such, BCMA has been investigated as a possible therapeutic target for multiple myeloma.

Accordingly, there is a need in the art for compositions that can be used in methods to treat multiple myeloma.

### SUMMARY

The subject specification provides anti-BCMA antibodies and antigen-binding fragments thereof.

In one aspect, an antibody or antigen-binding fragment thereof that specifically binds to BCMA is provided, comprising an antibody heavy chain variable (VH) domain and an antibody light chain variable (VL) domain, wherein the VH domain comprises a CDR-H1 sequence comprising the amino acid sequence of GFTFSNFGMH (SEQ ID NO: 1), a CDR-H2 sequence comprising the amino acid sequence of VIWSDETNR (SEQ ID NO: 2) and a CDR-H3 sequence comprising the amino acid sequence of DQQYCSSDSCFTWFDP (SEQ ID NO: 3), and the VL domain comprises a CDR-L1 sequence comprising the amino acid sequence of CX¹SSTGX²VTPX³X⁴YAN (SEQ ID NO: 25), wherein X¹ is R or A, X² is T or A, X³ is S or G, and X⁴ is N or Y, a CDR-L2 sequence comprising the amino acid sequence of DNNX⁵X⁶PP (SEQ ID NO: 26), wherein X⁵ is S, I, or N and X⁶ is R or K, and a CDR-L3 sequence comprising the amino acid sequence of ALX7X8GX9QWV (SEQ ID NO: 27), wherein X⁷ is W or Y, X³ is F or Y, and X⁹ is N or G.

In certain exemplary embodiments, the antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CASSTGTVTPSNYAN (SEQ ID NO: 4), CRSSTGTVTPSNYAN (SEQ ID NO: 5), CASSTGAVTPSNYAN (SEQ ID NO: 6), or CASSTGAVTPGYYAN (SEQ ID NO: 7), a CDR-L2 sequence comprising the amino acid sequence of DNNSRPP (SEQ ID NO: 9), DNNIKPP (SEQ ID NO: 10), or DNNNKPP (SEQ ID NO: 11), and a CDR-L3 sequence comprising the amino acid sequence of ALWFGNQWV (SEQ ID NO: 13), ALWYGGQWV (SEQ ID NO: 14), or ALYYGGQWV (SEQ ID NO: 15).

In certain exemplary embodiments, the antibody or antigen-binding fragment thereof comprises a VH domain that is at least about 90% identical or at least 95% identical to the amino acid sequence of SEQ ID NO: 16, and a VL domain that is at least about 90% identical or at least 95% identical to the amino acid sequence of SEQ ID NO: 17, SEQ ID NO: 18, SEQ ID NO: 19, SEQ ID NO: 20, SEQ ID NO: 21, SEQ ID NO: 23, or SEQ ID NO: 24.

In certain exemplary embodiments, the antibody or antigen-binding fragment thereof comprises an antibody heavy chain that is at least about 90% identical or at least 95% identical to the amino acid sequence of SEQ ID NO: 16, and an antibody light chain that is at least about 90% identical or at least 95% identical to the amino acid sequence of SEQ ID NO: 24.

In certain exemplary embodiments, the antibody or antigen-binding fragment thereof comprises a VH domain comprising the amino acid sequence of SEQ ID NO: 16, and a VL domain comprising an amino acid sequence selected from the group consisting of SEQ ID NO: 17, SEQ ID NO: 18, SEQ ID NO: 19, SEQ ID NO: 20, SEQ ID NO: 21, SEQ ID NO: 23, and SEQ ID NO: 24.

In certain exemplary embodiments, the antibody or antigen-binding fragment thereof comprises an antibody heavy chain comprising the amino acid sequence of SEQ ID NO: 16, and an antibody light chain comprising the amino acid sequence of SEQ ID NO: 24.

In certain exemplary embodiments, the antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CASSTGTVTPSNYAN (SEQ ID NO: 4), a CDR-L2 sequence comprising the amino acid sequence of DNNSRPP (SEQ ID NO: 9), and a CDR-L3 sequence comprising the amino acid sequence of ALWFGNQWV (SEQ ID NO: 13).

In certain exemplary embodiments, the antibody or antigen-binding fragment thereof has a heavy chain that is at least about 90% identical or at least 95% identical to the amino acid sequence of SEQ ID NO: 16, and a light chain that is at least about 90% identical or at least 95% identical to the amino acid sequence of SEQ ID NO: 18.

In certain exemplary embodiments, the antibody or antigen-binding fragment thereof comprises a heavy chain comprising the amino acid sequence of SEQ ID NO: 16, and a light chain comprising the amino acid sequence of SEQ ID NO: 18.

In certain exemplary embodiments, the antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CRSSTGTVTPSNYAN (SEQ ID NO: 5), a CDR-L2 sequence comprising the amino acid sequence of DNNSRPP (SEQ ID NO: 9), and a CDR-L3 sequence comprising the amino acid sequence of ALWFGNQWV (SEQ ID NO: 13).

In certain exemplary embodiments, the antibody or antigen-binding fragment comprises a heavy chain that is at least about 90% identical or at least 95% identical to the amino acid sequence of SEQ ID NO: 16, and a light chain that is at least about 90% identical or at least 95% identical to the amino acid sequence of SEQ ID NO: 19.

In certain exemplary embodiments, the antibody or antigen-binding fragment thereof comprises a heavy chain comprising the amino acid sequence of SEQ ID NO: 16, and a light chain comprising the amino acid sequence of SEQ ID NO: 19.

In certain exemplary embodiments, the antibody or antigen-binding fragment comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CASSTGTVTPSNYAN (SEQ ID NO: 4), a CDR-L2 sequence comprising the amino acid sequence of DNNSRPP (SEQ ID NO: 9), and a CDR-L3 sequence comprising the amino acid sequence of ALWFGNQWV (SEQ ID NO: 13).

In certain exemplary embodiments, the antibody or antigen-binding fragment thereof comprises an antibody heavy chain that is at least about 90% identical or at least 95% identical to the amino acid sequence of SEQ ID NO: 16, and an antibody light chain that is at least about 90% identical or at least 95% identical to the amino acid sequence of SEQ ID NO: 20.

In certain exemplary embodiments, the antibody or antigen-binding fragment thereof comprises an antibody heavy chain comprising the amino acid sequence of SEQ ID NO: 16, and an antibody light chain comprising the amino acid sequence of SEQ ID NO: 20.

In certain exemplary embodiments, the antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CASSTGAVTPSNYAN (SEQ ID NO: 6), a CDR-L2 sequence comprising the amino acid sequence of DNNIKPP (SEQ ID NO: 10), and a CDR-L3 sequence comprising the amino acid sequence of ALWYGGQWV (SEQ ID NO: 14).

In certain exemplary embodiments, the antibody or antigen-binding fragment thereof comprises an antibody heavy chain that is at least about 90% identical or at least 95% identical to the amino acid sequence of SEQ ID NO: 16, and an antibody light chain that is at least about 90% identical or at least 95% identical to the amino acid sequence of SEQ ID NO: 21.

In certain exemplary embodiments, the antibody or antigen-binding fragment thereof comprises an antibody heavy chain comprising the amino acid sequence of SEQ ID NO: 16, and an antibody light chain comprising the amino acid sequence of SEQ ID NO: 21.

In certain exemplary embodiments, the antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence CASSTGAVTPGYYAN (SEQ ID NO: 7), a CDR-L2 sequence comprising the amino acid sequence of DNNNKPP (SEQ ID NO: 11), and a CDR-L3 sequence comprising the amino acid sequence of ALYYGGQWV (SEQ ID NO: 15).

In certain exemplary embodiments, the antibody or antigen-binding fragment thereof comprises an antibody heavy chain that is at least about 90% identical or at least 95% identical to the amino acid sequence of SEQ ID NO: 16, and an antibody light chain that is at least about 90% identical or at least 95% identical to the amino acid sequence of SEQ ID NO: 23.

In certain exemplary embodiments, the antibody or antigen-binding fragment thereof comprises an antibody heavy chain comprising the amino acid sequence of SEQ ID NO: 16, and an antibody light chain comprising the amino acid sequence of SEQ ID NO: 23.

In certain exemplary embodiments, the antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CASSTGTVTPSNYAN (SEQ ID NO: 4), a CDR-L2 sequence comprising the amino acid sequence of DNNSRPP (SEQ ID NO: 9), and a CDR-L3 sequence comprising the amino acid sequence of ALWFGNQWV (SEQ ID NO: 13).

In certain exemplary embodiments, the antibody or antigen-binding fragment thereof is a chimeric or humanized antibody or antigen-binding fragment thereof.

In certain exemplary embodiments, the antibody or antigen-binding fragment thereof is a human antibody or antigen-binding fragment thereof.

In certain exemplary embodiments, the antibody or antigen-binding fragment thereof is a monoclonal antibody or antigen-binding fragment thereof.

In certain exemplary embodiments, the antibody or antigen-binding fragment thereof is a multi-specific antibody.

In certain exemplary embodiments, the antibody or antigen-binding fragment thereof comprises one or more full-length antibody heavy chains comprising an Fc region.

In certain exemplary embodiments, the Fc region is a human IgG1 Fc region.

In certain exemplary embodiments, the Fc region is a human IgG4 Fc region.

In certain exemplary embodiments, provided herein is a pharmaceutical composition comprising the antibody or antigen-binding fragment of the present disclosure and a pharmaceutically acceptable carrier.

In certain exemplary embodiments, provided herein is an isolated nucleic acid molecule encoding the antibody or antigen-binding fragment thereof.

In certain exemplary embodiments, provided herein is an expression vector comprising the nucleic acid molecule.

In certain exemplary embodiments, provided herein is a host cell comprising the nucleic acid molecule. In certain exemplary embodiments, provided herein is a host cell comprising the expression vector. In certain exemplary embodiments, provided herein is a host cell that is a mammalian cell.

In certain exemplary embodiments, provided herein is a method for making the antibody or antigen-binding fragment thereof, comprising culturing the host cell under suitable conditions and recovery of the antibody or antigen-binding fragment thereof.

In certain exemplary embodiments, provided herein is a method of treating or preventing a disease or disorder, the method comprising administering to a subject in thereof the pharmaceutical composition as provided herein.

In certain exemplary embodiments, provided herein is a method of treating or preventing a cancer, the method comprising administering to a subject in thereof the pharmaceutical composition as provided herein.

In certain exemplary embodiments, provided herein is a method of treating or preventing a plasm cell disorder, the method comprising administering to a subject in thereof the pharmaceutical composition as provided herein.

In certain exemplary embodiments, provided herein is a method for treating multiple myeloma in a subject, comprising administering to a subject in need thereof the antibody or antigen-binding fragment thereof.

In certain exemplary embodiments, the antibody or antigen-binding fragment thereof is for use as a medicament.

In certain exemplary embodiments, the antibody or antigen-binding fragment thereof is for use in a method for the treatment or prevention of a disease or disorder.

In certain exemplary embodiments, the antibody or antigen-binding fragment thereof is for use in a method for the treatment or prevention of a cancer.

In certain exemplary embodiments, the antibody or antigen-binding fragment thereof is for use in a method for the treatment or prevention of multiple myeloma.

In certain exemplary embodiments, the antibody or antigen-binding fragment thereof is for use in a method for the treatment or prevention of a plasma cell malignancy.

The summary of the disclosure described above is non-limiting and other features and advantages of the disclosed antigen-binding proteins and methods will be apparent from the following brief description of the drawings, detailed description of the disclosure, and claims.

### BRIEF DESCRIPTION OF THE DRAWINGS

The foregoing and other features and advantages of the present invention will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1A shows the seven anti-BCMA antibody VL domain variants and their percent sequence identity to the human germline VL.

FIG. 1B shows the sequence alignment of the parental VL domain and the seven VL domain variants.

FIG. 1C shows the sequence alignment of the CA10 parental VL domain with the human germline VL domain.

FIG. 2 is a table displaying the expression and purity of the anti-BCMA antibody variants. Purity was determined using SDS-PAGE and SEC.

FIG. 3 shows the cross-reactivity of the clone CA10 variants to human and cynomolgus BCMA proteins.

FIG. 4 shows binding between the anti-BCMA mAb antibodies and BCMA proteins on HEK cell membrane surfaces.

FIG. 5A-FIG. 5B show binding between the anti-BCMA mAb antibody variants (FIG. 5A) and the parental antibody (FIG. 5B) with BCMA proteins on MM1R cell membrane surfaces.

FIG. 6 shows the sensorgrams and affinity constants between the anti-BCMA mAb antibodies and human BCMA Avi-His protein.

FIG. 7 shows the sensorgrams and affinity constants between the anti-BCMA mAb antibodies and human BCMA DTA-His protein.

FIG. 8 shows the-sensorgrams and affinity constants between the anti-BCMA mAb antibodies and cynomolgus BCMA Avi-His protein.

FIG. 9 shows the sensorgrams and affinity constants between the anti-BCMA mAb antibodies and cynomolgus BCMA DTA-His protein.

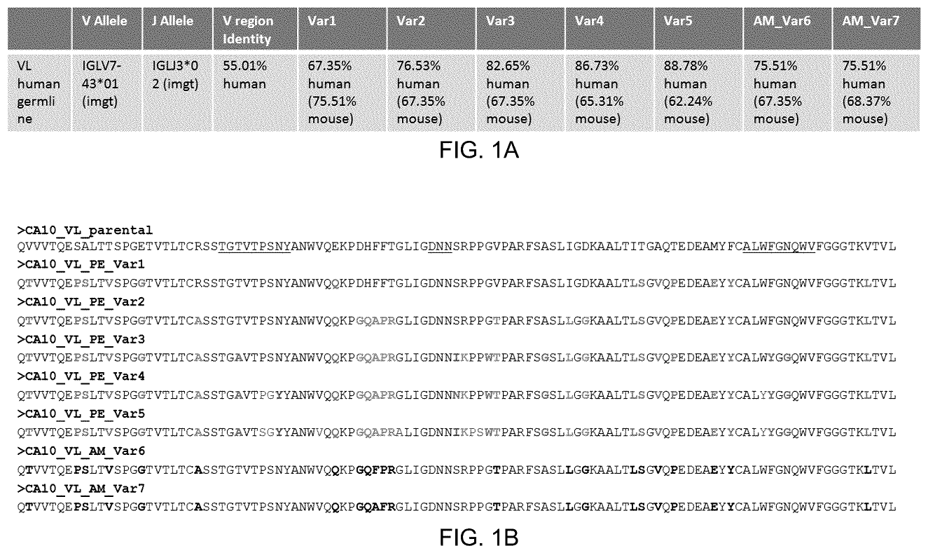

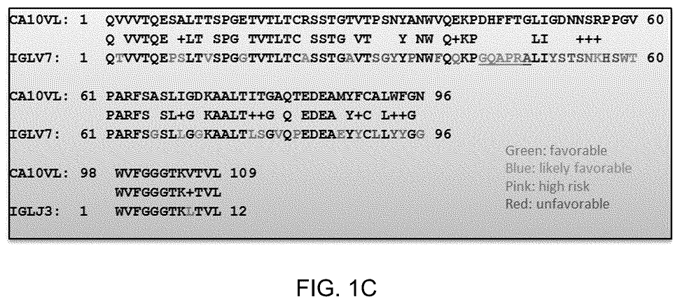

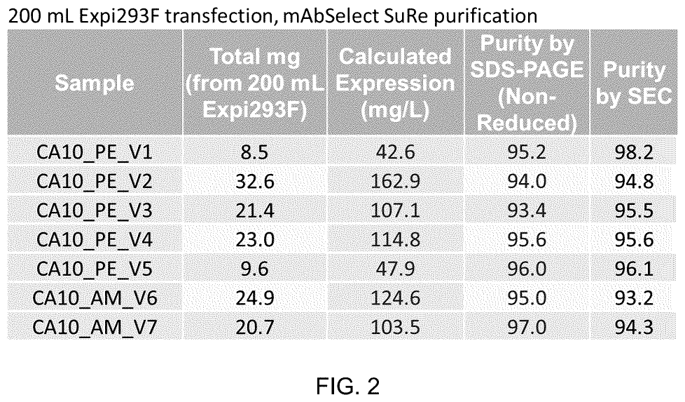

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

Although any methods and materials similar or equivalent to those described herein can be used in the practice of the present disclosure, exemplary methods and materials are now described. All publications mentioned herein are incorporated herein by reference to describe in their entirety.

As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells, and plasma cells. Its ligand is called B-cell activator of the TNF family (BAFF) and a proliferation inducing ligand (APRIL). BCMA is involved in mediating the survival of plasma cells for maintaining long-term humoral immunity. The gene for BCMA is encoded on chromosome 16 producing a primary mRNA transcript of 994 nucleotides in length (NCBI accession NM_001192.2) that encodes a protein of 184 amino acids (NP_001183.2). A second antisense transcript derived from the BCMA locus has been described, which may play a role in regulating BCMA expression. (Laabi Y. et al., Nucleic Acids Res., 1994, 22:1147-1154). Additional transcript variants have been described with unknown significance (Smirnova A S et al. Mol Immunol., 2008, 45(4):1179-1183. A second isoform, also known as TV4, has been identified (Uniprot identifier Q02223-2). As used herein, “BCMA” includes proteins comprising mutations, e.g., point mutations, fragments, insertions, deletions and splice variants of full length wild-type BCMA.

As used herein, the term “antibody” or “antigen-binding protein” refers to an immunoglobulin molecule that specifically binds to, or is immunologically reactive with an antigen or epitope (e.g., a BCMA antigen or epitope), and includes both polyclonal and monoclonal antibodies, as well as functional antibody fragments thereof, including but not limited to fragment antigen-binding (Fab) fragments, F(ab′)₂ fragments, Fab′ fragments, Fv fragments, recombinant IgG (rlgG) fragments, single chain variable fragments (scFv) and single domain antibodies (e.g., sdAb, sdFv, nanobody) fragments. The term “antibody” includes genetically engineered or otherwise modified forms of immunoglobulins, such as intrabodies, peptibodies, chimeric antibodies, fully human antibodies, humanized antibodies, meditope-enabled antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments thereof. As used herein, the term “functional antibody fragment” refers to an antibody fragment having at least 80%, at least 85%, at least 90%, or at least 95% affinity as the antibody of interest from which the fragment is derived from.

As used herein, the term “complementarity determining region” or “CDR” refers to sequences of amino acids within antibody variable regions, which confer antigen specificity and binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (AHo numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

A “CDR” or “complementary determining region,” or individual specified CDRs (e.g., “CDR-H1,” “CDR-H2,” “CDR-H3”), of a given antibody or region thereof, such as a variable region thereof, should be understood to encompass a (or the specific) complementary determining region as defined by any of the known schemes. Likewise, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the IMGT, Kabat, Chothia, AbM, or Contact method. In other cases, the particular amino acid sequence of a CDR or FR is given. Unless otherwise specified, all particular CDR amino acid sequences mentioned in the disclosure are IMGT CDRs. However, alternative CDRs defined by other schemes are also encompassed by the present disclosure, such as those determined by abYsis Key Annotation (Website: abysis.org/abysis/sequence_input/key_annotation/key_annotation.cgi).

The term “human antibody,” as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “multispecific antigen-binding molecules,” as used herein refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. A multispecific antigen-binding molecule can be a single multifunctional polypeptide, or it can be a multimeric complex of two or more polypeptides that are covalently or non-covalently associated with one another. The term “multispecific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In certain exemplary embodiments, an antibody of the present disclosure is functionally linked to another antibody or antigen-binding fragment thereof to produce a bispecific antibody with a second binding specificity.

As used herein, the term “specifically binds,” “specifically binding,” “binding specificity” or “specifically recognized” refers that an antigen binding protein or antigen-binding fragment thereof that exhibits appreciable affinity for an antigen (e.g., a BCMA antigen) and does not exhibit significant cross reactivity to a target that is not a BCMA protein. As used herein, the term “affinity” refers to the strength of the interaction between an antigen binding protein or antigen-binding fragment thereof antigen binding site and the epitope to which it binds. In certain exemplary embodiments, affinity is measured by surface plasmon resonance (SPR), e.g., in a Biacore instrument. As readily understood by those skilled in the art, an antigen binding protein affinity may be reported as a dissociation constant (KD) in molarity (M). The antigen binding protein or antigen-binding fragment thereof of the disclosure have KD values in the range of about 10⁻⁶ M to about 10⁻¹² M (i.e., low micromolar to picomolar range), about 10⁻⁷ M to 10⁻¹¹ M, about 10⁻⁸ M to about 10⁻¹⁰ M, about 10⁻⁹ M. In certain embodiments, the antigen binding protein or antigen-binding fragment thereof has a binding affinity of about 10⁻⁶ M, 10⁻⁷ M, 10⁻⁸ M, 10⁻⁹ M, 10⁻¹⁰ M, 10⁻¹¹ M, or 10⁻¹² M. In certain embodiments, the antigen binding protein or antigen-binding fragment thereof has a binding affinity of about 10⁻⁷ M to about 10⁻⁹ M (nanomolar range).

Specific binding can be determined according to any art-recognized means for determining such binding. In some embodiments, specific binding is determined by competitive binding assays (e.g. ELISA) or Biacore assays. In certain embodiments, the assay is conducted at about 20° C., 25° C., 30° C., or 37° C.

As used herein, “administer” or “administration” refers to the act of injecting or otherwise physically delivering a substance as it exists outside the body (e.g., an isolated binding polypeptide provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “about” or “approximately” means within about 20%, such as within about 10%, within about 5%, or within about 1% or less of a given value or range.

### Anti-BCMA Antibodies

In one aspect, the disclosure provides antibodies or antigen-binding fragments thereof with binding specificity to BCMA.

Exemplary anti-BCMA antibody or antigen-binding fragment thereof CDRs are recited below in Table 1. Exemplary anti-BCMA antibody or antigen-binding fragment thereof variable heavy (VH) and variable light (VL) domains are recited below in Table 2.

TABLE 1
Antibody heavy chain and light chain CDR regions
Heavy Chain

| VH Chain ID | HCDR1 | HCDR2 | HCDR3 |
|---|---|---|---|
| Anti-BCMA VH | GFTFSNFGMH | VIWSDETNR | DQQYCSSDSCFTWFDP |
| | (SEQ ID NO: 1) | (SEQ ID NO: 2) | (SEQ ID NO: 3) |

Light Chain

| VL Chain ID | LCDR1 | LCDR2 | LCDR3 |
|---|---|---|---|
| Anti-BCMA | CRSSTGTVTPSNYAN | DNNSRPP | ALWFGNQWV |
| VL_parent | (SEQ ID NO: 5) | (SEQ ID NO: 9) | (SEQ ID NO: 13) |
| Anti-BCMA VL_v1 | CRSSTGTVTPSNYAN | DNNSRPP | ALWFGNQWV |
| | (SEQ ID NO: 5) | (SEQ ID NO: 9) | (SEQ ID NO: 13) |
| Anti-BCMA VL_v2 | CASSTGTVTPSNYAN | DNNSRPP | ALWFGNQWV |
| | (SEQ ID NO: 4) | (SEQ ID NO: 9) | (SEQ ID NO: 13) |
| Anti-BCMA VL_v3 | CASSTGAVTPSNYAN | DNNIKPP | ALWYGGQWV |
| | (SEQ ID NO: 6) | (SEQ ID NO: 10) | (SEQ ID NO: 14) |
| Anti-BCMA VL_v4 | CASSTGAVTPGYYAN | DNNNKPP | ALYYGGQWV |
| | (SEQ ID NO: 7) | (SEQ ID NO: 11) | (SEQ ID NO: 15) |
| Anti-BCMA VL_v5 | CASSTGAVTSGYYAN | DNNIKPS | ALYYGGQWV |
| | (SEQ ID NO: 8) | (SEQ ID NO: 12) | (SEQ ID NO: 15) |
| Anti-BCMA VL_v6 | CASSTGTVTPSNYAN | DNNSRPP | ALWFGNQWV |
| | (SEQ ID NO: 4) | (SEQ ID NO: 9) | (SEQ ID NO: 13) |
| Anti-BCMA VL_v7 | CASSTGTVTPSNYAN | DNNSRPP | ALWFGNQWV |
| | (SEQ ID NO: 4) | (SEQ ID NO: 9) | (SEQ ID NO: 13) |

TABLE 2
Antibody VH and VL sequences

| Antibody ID | Sequence |
|---|---|
| Anti-BCMA VH | QVQLVESGGGVVQPGRSLRLSCAASGFTFSNFGMHWVRQAPGRGLEWVAVIWS |
| | DETNRYYADSVKGRFTVSRDNVKSTVYLQMNSLISEDTAVYYCARDQQYCSSDSC |
| | FTWFDPWGQGTLVTVSS (SEQ ID NO: 16) |
| Anti-BCMA | QVVVTQESALTTSPGETVTLTCRSSTGTVTPSNYANWVQEKPDHFFTGLIGDNNSR |
| VL_parent | PPGVPARFSASLIGDKAALTITGAQTEDEAMYFCALWFGNQWVFGGGTKVTVL |
| | (SEQ ID NO: 17) |
| Anti-BCMA | QTVVTQEPSLTVSPGGTVTLTCRSSTGTVTPSNYANWVQQKPDHFFTGLIGDNNS |
| VL_v1 | RPPGVPARFSASLIGDKAALTLSGVQPEDEAEYYCALWFGNQWVFGGGTKLTVL |
| | (SEQ ID NO: 18) |
| Anti-BCMA | QTVVTQEPSLTVSPGGTVTLTCASSTGTVTPSNYANWVQQKPGQAPRGLIGDNNS |
| VL_v2 | RPPGTPARFSASLLGGKAALTLSGVQPEDEAEYYCALWFGNQWVFGGGTKLTVL |
| | (SEQ ID NO: 19) |
| Anti-BCMA | QTVVTQEPSLTVSPGGTVTLTCASSTGAVTPSNYANWVQQKPGQAPRGLIGDNNI |
| VL_v3 | KPPWTPARFSGSLLGGKAALTLSGVQPEDEAEYYCALWYGGQWVFGGGTKLTVL |
| | (SEQ ID NO: 20) |
| Anti-BCMA | QTVVTQEPSLTVSPGGTVTLTCASSTGAVTPGYYANWVQQKPGQAPRGLIGDNNN |
| VL_v4 | KPPWTPARFSGSLLGGKAALTLSGVQPEDEAEYYCALYYGGQWVFGGGTKLTVL |
| | (SEQ ID NO: 21) |
| Anti-BCMA | QTVVTQEPSLTVSPGGTVTLTCASSTGAVTSGYYANWVQQKPGQAPRALIGDNNI |
| VL_v5 | KPSWTPARFSGSLLGGKAALTLSGVQPEDEAEYYCALYYGGQWVFGGGTKLTVL |
| | (SEQ ID NO: 22) |
| Anti-BCMA | QTVVTQEPSLTVSPGGTVTLTCASSTGTVTPSNYANWVQQKPGQFPRGLIGDNNS |
| VL_v6 | RPPGTPARFSASLLGGKAALTLSGVQPEDEAEYYCALWFGNQWVFGGGTKLTVL |
| | (SEQ ID NO: 23) |
| Anti-BCMA | QTVVTQEPSLTVSPGGTVTLTCASSTGTVTPSNYANWVQQKPGQAFRGLIGDNNS |
| VL_v7 | RPPGTPARFSASLLGGKAALTLSGVQPEDEAEYYCALWFGNQWVFGGGTKLTVL |
| | (SEQ ID NO: 24) |

In certain embodiments, the anti-BCMA antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2) and a CDR-H3 sequence comprising the amino acid sequence of DQQYCSSDSCFTWFDP (SEQ ID NO: 3), and a VL domain comprising a CDR-L1 sequence comprising the amino acid sequence of CX¹SSTGX²VTPX³X⁴YAN (SEQ ID NO: 25), wherein X¹ is R or A, X² is T or A, X³ is S or G, and X⁴ is N or Y, a CDR-L2 sequence comprising the amino acid sequence of DNNX⁵X⁶PP (SEQ ID NO: 26), wherein X⁵ is S, I, or N and X⁶ is R or K, and a CDR-L3 sequence comprising the amino acid sequence of ALX7X8GX9QWV (SEQ ID NO: 27), wherein X⁷ is W or Y, X³ is F or Y, and X⁹ is N or G.

In certain embodiments, the anti-BCMA antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CASSTGTVTPSNYAN (SEQ ID NO: 4), CRSSTGTVTPSNYAN (SEQ ID NO: 5), CASSTGAVTPSNYAN (SEQ ID NO: 6), or CASSTGAVTPGYYAN (SEQ ID NO: 7), a CDR-L2 sequence comprising the amino acid sequence of DNNSRPP (SEQ ID NO: 9), DNNIKPP (SEQ ID NO: 10), or DNNNKPP (SEQ ID NO: 11), and a CDR-L3 sequence comprising the amino acid sequence of ALWFGNQWV (SEQ ID NO: 13), ALWYGGQWV (SEQ ID NO: 14), or ALYYGGQWV (SEQ ID NO: 15).

In certain embodiments, the anti-BCMA antibody or antigen-binding fragment thereof comprises a VH domain comprising a CDR-H1 sequence comprising the amino acid sequence of GFTFSNFGMH (SEQ ID NO: 1), a CDR-H2 sequence comprising the amino acid sequence of VIWSDETNR (SEQ ID NO: 2), and a CDR-H3 sequence comprising the amino acid sequence of DQQYCSSDSCFTWFDP (SEQ ID NO: 3), and a VL domain comprising a CDR-L1 sequence comprising the amino acid sequence of CASSTGTVTPSNYAN (SEQ ID NO: 4, a CDR-L2 sequence comprising the amino acid sequence of DNNSRPP (SEQ ID NO: 9), and a CDR-L3 sequence comprising the amino acid sequence of ALWFGNQWV (SEQ ID NO: 13).

In certain embodiments, the anti-BCMA antibody or antigen-binding fragment thereof comprises a VH domain that is at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more identical to the amino acid sequence of SEQ ID NO: 16, and a VL domain that is at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more identical to the amino acid sequence of SEQ ID NO: 17, SEQ ID NO: 18, SEQ ID NO: 19, SEQ ID NO: 20, SEQ ID NO: 21, SEQ ID NO: 23, or SEQ ID NO: 24.

In certain embodiments, the anti-BCMA antibody or antigen-binding fragment thereof comprises a VH domain that is at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more identical to the amino acid sequence of SEQ ID NO: 16, and a VL domain that is at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more identical to the amino acid sequence of SEQ ID NO: 24.

In certain embodiments, the anti-BCMA antibody or antigen-binding fragment thereof comprises a VH domain comprising the amino acid sequence of SEQ ID NO: 16, and a VL domain comprising an amino acid sequence selected from the group consisting of SEQ ID NO: 17, SEQ ID NO: 18, SEQ ID NO: 19, SEQ ID NO: 20, SEQ ID NO: 21, SEQ ID NO: 23, and SEQ ID NO: 24.

In certain embodiments, the anti-BCMA antibody or antigen-binding fragment thereof comprises an antibody heavy chain comprising the amino acid sequence of SEQ ID NO: 16, and an antibody light chain comprising the amino acid sequence of SEQ ID NO: 2.

In certain embodiments, the anti-BCMA antibody or antigen-binding fragment thereof is a chimeric or humanized antibody or antigen-binding fragment thereof. In certain embodiments, the antibody or antigen-binding fragment thereof is a human antibody or antigen-binding fragment thereof.

In certain embodiments, the anti-BCMA antibody or antigen-binding fragment thereof is a monoclonal antibody or antigen-binding fragment thereof.

In certain embodiments, the anti-BCMA antibody or antigen-binding fragment thereof is an anti-BCMA antibody-drug conjugate.

In certain embodiments, the anti-BCMA antibody or antigen-binding fragment thereof is a monospecific antibody.

In certain embodiments, the anti-BCMA antibody or antigen-binding fragment thereof is a bispecific antibody.

In certain embodiments, the anti-BCMA antibody or antigen-binding fragment thereof is a trispecific antibody.

In certain embodiments, the anti-BCMA antibody or antigen-binding fragment thereof is a multi-specific antibody.

### Immune Cell Engagers

The anti-BCMA antigen-binding proteins of the disclosure may be present in an immune cell engaging format. An immune cell engager is an antigen-binding protein that comprises at least one binding domain capable of binding to a cell surface protein of an immune cell and at least one binding domain to a separate cell surface protein (e.g., a surface protein on a tumor cell, such as BCMA). The binding domain specific to the cell surface protein of an immune cell is capable of recruiting immune cells specifically to the target tumor cells to be eliminated. Examples of immune cells that may be recruited include, but are not limited to, T cells, B cells, natural killer (NK) cells, natural killer T (NKT) cells, neutrophil cells, monocytes, and macrophages. Examples of surface proteins that may be used to recruit immune cells includes, but are limited to, CD3, TCRa, TCRp, CD16, NKG2D, CD89, CD64, and CD32a.

### Expression of Antigen-Binding Proteins

In one aspect, polynucleotides encoding the binding proteins (e.g., antigen-binding proteins and antigen-binding fragments thereof) disclosed herein are provided. Methods of making binding proteins comprising expressing these polynucleotides are also provided.

Polynucleotides encoding the binding proteins disclosed herein are typically inserted in an expression vector for introduction into host cells that may be used to produce the desired quantity of the binding proteins. Accordingly, in certain aspects, the disclosure provides expression vectors comprising polynucleotides disclosed herein and host cells comprising these vectors and polynucleotides.

The term “vector” or “expression vector” is used herein to mean vectors used in accordance with the present disclosure as a vehicle for introducing into and expressing a desired gene in a cell. As known to those skilled in the art, such vectors may readily be selected from the group consisting of plasmids, phages, viruses and retroviruses. In general, vectors compatible with the disclosure will comprise a selection marker, appropriate restriction sites to facilitate cloning of the desired gene and the ability to enter and/or replicate in eukaryotic or prokaryotic cells.

Numerous expression vector systems may be employed for the purposes of this disclosure. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding a binding protein, e.g. an antibody or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, from supernatant of lysed cells culture, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HEK (human kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT® cells) (Biowa, Princeton, N.J.)). In one embodiment, NSO cells may be used. CHO cells are particularly useful. Host cell lines are typically available from commercial services, e.g., the American Tissue Culture Collection, or from authors of published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the binding proteins featured in the disclosure can also be expressed in non-mammalian cells such as bacteria or yeast or plant cells. In this regard, it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the binding proteins can become part of inclusion bodies. In some embodiments, the binding proteins are then isolated, purified and assembled into functional molecules. In some embodiments, the binding proteins of the disclosure are expressed in a bacterial host cell. In some embodiments, the bacterial host cell is transformed with an expression vector comprising a nucleic acid molecule encoding a binding protein of the disclosure.

In addition to prokaryotes, eukaryotic microbes may also be used. Saccharomyces cerevisiae, or common baker's yeast, is the most commonly used among eukaryotic microbes, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### Methods of Administering Antigen Binding Proteins

Methods of preparing and administering antigen binding proteins (e.g., the anti-BCMA antibody or antigen binding fragment thereof disclosed herein) to a subject are well known to or are readily determined by those skilled in the art. The route of administration of the antigen binding proteins of the current disclosure may be oral, parenteral, by inhalation or topical. The term parenteral as used herein includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the modified antibodies can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M or 0.05 M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage, and should also be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. Isotonic agents, for example, sugars, polyalcohols, such as mannitol, sorbitol, or sodium chloride may also be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation typically include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture can include labels or package inserts indicating that the associated compositions are useful for treating a subject suffering from, or predisposed to autoimmune or neoplastic disorders.

Effective doses of the compositions of the present disclosure, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For passive immunization with antigen binding proteins, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example, dosages can be 1 mg/kg body weight or 10 mg/kg body weight or within the range of 1-10 mg/kg, e.g., at least 1 mg/kg. Doses intermediate in the above ranges are also intended to be within the scope of the current disclosure. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimens entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more antigen binding proteins with different binding specificities are administered simultaneously, in which case the dosage of each antigen binding protein administered falls within the ranges indicated.

Antigen binding proteins described herein can be administered on multiple occasions. Intervals between single dosages can be weekly, monthly or yearly. Intervals can also be irregular as indicated by measuring blood levels of modified binding polypeptide or antigen in the patient. In some methods, dosage is adjusted to achieve a plasma modified antigen binding protein concentration of 1-1000 μg/ml and in some methods 25-300 μg/ml. Alternatively, antigen binding protein can be administered as a sustained release formulation, in which case less frequent administration is required. For antigen binding proteins, dosage and frequency vary depending on the half-life of the antigen binding protein in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present antigen binding protein or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of antibody per dose, with dosages of from 5 to 25 mg being more commonly used for radioimmunoconjugates and higher doses for cytotoxin-drug modified antibodies) at relatively short intervals is sometimes required until progression of the disease is reduced or terminated, or until the patient shows partial or complete amelioration of disease symptoms. Thereafter, the patient can be administered a prophylactic regime.

Antigen binding proteins described herein can optionally be administered in combination with other agents that are effective in treating the disorder or condition in need of treatment (e.g., prophylactic or therapeutic). Effective single treatment dosages (i.e., therapeutically effective amounts) of ⁹⁰Y-labeled modified antibodies of the current disclosure range from between about 5 and about 75 mCi, such as between about 10 and about 40 mCi. Effective single treatment non-marrow ablative dosages of ¹³¹I-modified antibodies range from between about 5 and about 70 mCi, such as between about 5 and about 40 mCi. Effective single treatment ablative dosages (i.e., may require autologous bone marrow transplantation) of ¹³¹I-labeled antibodies range from between about 30 and about 600 mCi, such as between about 50 and less than about 500 mCi. In conjunction with a chimeric antibody, owing to the longer circulating half-life vis-a-vis murine antibodies, an effective single treatment non-marrow ablative dosages of ¹³¹I labeled chimeric antibodies range from between about 5 and about 40 mCi, e.g., less than about 30 mCi. Imaging criteria for, e.g., an ¹¹¹In label, are typically less than about 5 mCi.

While the antigen binding proteins may be administered as described immediately above, it must be emphasized that in other embodiments antigen binding proteins may be administered to otherwise healthy patients as a first line therapy. In such embodiments the antigen binding proteins may be administered to patients having normal or average red marrow reserves and/or to patients that have not, and are not, undergoing one or more other therapies. As used herein, the administration of modified antibodies or fragments thereof in conjunction or combination with an adjunct therapy means the sequential, simultaneous, coextensive, concurrent, concomitant, or contemporaneous administration or application of the therapy and the disclosed antibodies. Those skilled in the art will appreciate that the administration or application of the various components of the combined therapeutic regimen may be timed to enhance the overall effectiveness of the treatment. A skilled artisan (e.g. an experienced oncologist) would readily be able to discern effective combined therapeutic regimens without undue experimentation based on the selected adjunct therapy and the teachings of the instant specification.

As previously discussed, the antigen binding proteins of the present disclosure, immunoreactive fragments or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the modified antigen binding proteins, immunoreactive fragment or recombinant thereof, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the modified binding polypeptide will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

The biological activity of the pharmaceutical compositions defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used. Furthermore, computer-aided tomography, X-ray, nuclear magnetic resonance tomography (e.g. for National Cancer Institute-criteria based response assessment [Cheson B D, Horning S J, Coiffier B, Shipp M A, Fisher R I, Connors J M, Lister T A, Vose J, Grillo-Lopez A, Hagenbeek A, Cabanillas F, Klippensten D, Hiddemann W, Castellino R, Harris N L, Armitage J O, Carter W, Hoppe R, Canellos G P. Report of an international workshop to standardize response criteria for non-Hodgkin's lymphomas. NCI Sponsored International Working Group. J Clin Oncol. 1999 April; 17(4):1244]), positron-emission tomography scanning, white blood cell counts, differentials, Fluorescence Activated Cell Sorting, bone marrow aspiration, lymph node biopsies/histologies, and various lymphoma specific clinical chemistry parameters (e.g. lactate dehydrogenase) and other established standard methods may be used.

### Examples

The following examples are put forth so as to provide those of ordinary skill in the art with a complete disclosure and description of how to make and use the methods and compositions featured in the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Anti-BCMA Antibodies

Generation of Anti-Human BCMA Antibodies

A cohort of 15 eight-week female Trianni human Ig transgenic mice were implanted with an Estradiol pellet subcutaneously and after three weeks injected with 100 μg of anti-mouse CD25 antibody. A week later, all mice were primed with intraperitoneal administration of approximately 5×10⁶ (5 million) 300.19 cells overexpressing human BCMA in PBS without adjuvant. This was followed by three immunizations with extracellular domain (ECD) of human BCMA conjugated to Diphtheria toxin A (DTA) protein and two immunizations with ECD of cynomolgus macaques conjugated to Diphtheria toxin A (DTA) protein in sigma adjuvant. Anti-BCMA specific IgG titer in blood was measured by ELISA after the last immunization using the human BCMA-ECD protein as antigen to assess the immune responses. Mice that developed high anti-BCMA antibody titers (1:100000 or more) were selected for hybridoma generation. These mice were boosted with a combination of ECD domains of human & cynomolgus BCMA-DTA protein without adjuvant four days before the mice were sacrificed for harvesting spleen for preparation of fusion.

The process of fusion and generation of hybridomas was performed following established protocols (Yale J Biol Med, 1981, 54 (5) 387-402) and Gefter et al. (Somatic Cell Genet, 1977, 3 (2) 231-236). Single cell suspension of splenocytes were mixed with FO myeloma cells at a ratio of 5:1 in a 50 ml conical polypropylene tube and cells were washed twice with serum-free IMDM. To this, 1 mL of polyethylene glycol 1500 (PEG) (Roche Applied Science, Indianapolis, IN) preheated at 37° C. was added slowly to cell pellet over the course of about 1 minute, while gently rocking the tube. The cells were incubated in the PEG for one minute followed by addition of one mL serum-free IMDM added dropwise to pellet over the course of 30 seconds, and then 9 mL of serum-free IMDM were added to pellet for one minute. The tube was centrifuged at 350×g for 10 minutes at room temperature, and the supernatant was aspirated. The pellet was resuspended in 200 mL of filtered complete hybridoma production media, IMDM (Hyclone) supplemented with 10% FBS (Hyclone), 1× non-essential amino acid (Gibco), 1 mM sodium pyruvate (Gibco), 1× penicillin-streptomycin (Gibco), Hybridoma Fusion and Cloning Factor (Roche), and 1×HAT (Sigma). The cells were transferred to a T-150 suspension cell flask and kept in an incubator at 37° C., 7% CO₂.

The following day, the cells in the T-150 flask were centrifuged and the supernatants removed. The cells were carefully transferred to hybridoma semi-solid selection and cloning media (Molecular Devices) containing FITC conjugated anti-mouse IgG (Molecular Devices). The cells were gently mixed, and the semi-solid medium was seeded into six well plates at 2 mL/well. These plates were incubated at 37° C., 7% CO₂ for the growth of hybridomas. After about 10 days of incubation, hybridomas secreting antibodies react with FITC conjugated anti-mouse IgG and create a green halo surrounding the hybridoma. These hybridomas were detected by Clonepix (Molecular devices) and single colonies were picked into wells of multiple 96 well plates. The 96 well plates containing clonal hybridomas were incubated for an additional 10 days at 37° C., 7% CO₂.

Anti-BCMA Antibody Sequences

The heavy chain variable region is set forth as:
QVQLVESGGGVVQPGRSLRLSCAASGFTFSNFGMHWVRQAPGRGLEWVAVIWSDETNRYYA
DSVKGRFTVSRDNVKSTVYLQMNSLISEDTAVYYCARDQQYCSSDSCFTWFDPWGQGTLVTV
SS (SEQ ID NO: 16). Underlined residue sections are CDRs 1, 2, and 3.
CDR-H1 is set forth as GFTFSNFGMH (SEQ ID NO: 1). CDR-H2 is set forth as VIWSDETNR
(SEQ ID NO: 2). CDR-H3 is set forth as DQQYCSSDSCFTWFDP (SEQ ID NO: 3).
The parental light chain variable region is set forth as:
QVVVTQESALTTSPGETVTLTCRSSTGTVTPSNYANWVQEKPDHFFTGLIGDNNSRPPGVPAR
FSASLIGDKAALTITGAQTEDEAMYFCALWFGNQWVFGGGTKVTVL (SEQ ID NO: 17).
Underlined residue sections are CDRs 1, 2, and 3.
CDR-L1 is set forth as CRSSTGTVTPSNYAN (SEQ ID NO: 5). CDR-L2 is set forth as
DNNSRPP (SEQ ID NO: 9). CDR-L3 is set forth as ALWFGNQWV (SEQ ID NO: 13).
The light chain variable region of Variant 1 is set forth as:
QTVVTQEPSLTVSPGGTVTLTCRSSTGTVTPSNYANWVQQKPDHFFTGLIGDNNSRPPGVPA
RFSASLIGDKAALTLSGVQPEDEAEYYCALWFGNQWVFGGGTKLTVL (SEQ ID NO: 18).
Underlined residue sections are CDRs 1, 2, and 3.
CDR-L1 is set forth as CRSSTGTVTPSNYAN (SEQ ID NO: 5). CDR-L2 is set forth as
DNNSRPP (SEQ ID NO: 9). CDR-L3 is set forth as ALWFGNQWV (SEQ ID NO: 13).
The light chain variable region of Variant 2 is set forth as:
QTVVTQEPSLTVSPGGTVTLTCASSTGTVTPSNYANWVQQKPGQAPRGLIGDNNSRPPGTPA
RFSASLLGGKAALTLSGVQPEDEAEYYCALWFGNQWVFGGGTKLTVL (SEQ ID NO: 19).
Underlined residue sections are CDRs 1, 2, and 3.
CDR-L1 is set forth as CASSTGTVTPSNYAN (SEQ ID NO: 4). CDR-L2 is set forth as
DNNSRPP (SEQ ID NO: 9). CDR-L3 is set forth as ALWFGNQWV (SEQ ID NO: 13).
The light chain variable region of Variant 3 is set forth as:
QTVVTQEPSLTVSPGGTVTLTCASSTGAVTPSNYANWVQQKPGQAPRGLIGDNNIKPPWTPA
RFSGSLLGGKAALTLSGVQPEDEAEYYCALWYGGQWVFGGGTKLTVL (SEQ ID NO: 20).
Underlined residue sections are CDRs 1, 2, and 3.
CDR-L1 is set forth as CASSTGAVTPSNYAN (SEQ ID NO: 6). CDR-L2 is set forth as
DNNIKPP (SEQ ID NO: 10). CDR-L3 is set forth as ALWYGGQWV (SEQ ID NO: 14).
The light chain variable region of Variant 4 is set forth as:
QTVVTQEPSLTVSPGGTVTLTCASSTGAVTPGYYANWVQQKPGQAPRGLIGDNNNKPPWTPA
RFSGSLLGGKAALTLSGVQPEDEAEYYCALYYGGQWVFGGGTKLTVL (SEQ ID NO: 21).
Underlined residue sections are CDRs 1, 2, and 3.
CDR-L1 is set forth as CASSTGAVTPGYYAN (SEQ ID NO: 7). CDR-L2 is set forth as
DNNNKPP (SEQ ID NO: 11). CDR-L3 is set forth as ALYYGGQWV (SEQ ID NO: 15).
The light chain variable region of Variant 5 is set forth as:
QTVVTQEPSLTVSPGGTVTLTCASSTGAVTSGYYANWVQQKPGQAPRALIGDNNIKPSWTPA
RFSGSLLGGKAALTLSGVQPEDEAEYYCALYYGGQWVFGGGTKLTVL (SEQ ID NO: 22).
Underlined residue sections are CDRs 1, 2, and 3.
CDR-L1 is set forth as CASSTGAVTSGYYAN (SEQ ID NO: 8). CDR-L2 is set forth as
DNNIKPS (SEQ ID NO: 12). CDR-L3 is set forth as ALYYGGQWV (SEQ ID NO: 15).
The light chain variable region of Variant 6 is set forth as:
QTVVTQEPSLTVSPGGTVTLTCASSTGTVTPSNYANWVQQKPGQFPRGLIGDNNSRPPGTPA
RFSASLLGGKAALTLSGVQPEDEAEYYCALWFGNQWVFGGGTKLTVL (SEQ ID NO: 23).
Underlined residue sections are CDRs 1, 2, and 3.
CDR-L1 is set forth as CASSTGTVTPSNYAN (SEQ ID NO: 4). CDR-L2 is set forth as
DNNSRPP (SEQ ID NO: 9). CDR-L3 is set forth as ALWFGNQWV (SEQ ID NO: 13).
The light chain variable region of Variant 7 is set forth as:
QTVVTQEPSLTVSPGGTVTLTCASSTGTVTPSNYANWVQQKPGQAFRGLIGDNNSRPPGTPA
RFSASLLGGKAALTLSGVQPEDEAEYYCALWFGNQWVFGGGTKLTVL (SEQ ID NO: 24).
Underlined residue sections are CDRs 1, 2, and 3.
CDR-L1 is set forth as CASSTGTVTPSNYAN (SEQ ID NO: 4). CDR-L2 is set forth as
DNNSRPP (SEQ ID NO: 9). CDR-L3 is set forth as ALWFGNQWV (SEQ ID NO: 13).

### Example 2. Expression and Purification of Anti-BCMA Antibodies

Expi293F™ cells were subcultured until cell density reached about 3-5×10⁶ viable cells/mL. Cells were seeded to a final density of 2.5-3×10⁶ viable cells/mL, then were allowed to grow overnight. Then, cells were diluted to a final density of 3×10⁶ viable cells/mL with fresh, pre-warmed Expi293™ Expression Medium, and cells were gently mixed.

Plasmid DNA was diluted with OPTI-PLEX™ Complexation Buffer (or OPTI-MEM™ I Medium) to a total plasmid DNA concentration of about 1.0 μg/mL. EXPIFECTAMINE™ 293 Reagent was diluted with diluted with OPTI-PLEX™ Complexation Buffer (or OPTI-MEM™ I Medium). Both solutions were incubated at room temperature for 5 minutes. The diluted EXPIFECTAMINE™ 293 Reagent was added to the diluted plasmid DNA, and the solution was mixed, and the solution containing the EXPIFECTAMINE™ 293/plasmid DNA complexes was incubated at room temperature for 10⁻²⁰ minutes. The complexes were slowly transferred to the Expi293F™ cells with gentle swirling during the addition, then the cells were incubated in a 37° C. incubator with ≥80% relative humidity and 8% CO₂ on an orbital shaker.

EXPIFECTAMINE™ 293 Transfection Enhancer 1 and EXPIFECTAMINE™ 293 Transfection Enhancer 2 were added to the transfection flask about 18-22 hours post-transfection, the flask was gently swirled during addition, then the flask was immediately returned to the 37° C. incubator with ≥80% relative humidity and 8% CO₂ on an orbital shaker platform.

The protein containing culture supernatant was harvested between about 5 to 7 days post-transfection.

### Purification

The antibodies were purified on a mAbSelect SuRe column (GE #11-0034-95, 16 mm×25 mm). Prior to loading onto the column, the antibody solutions were filtered through a 0.2 μm PES filter unit to remove any particulates. The following buffers were used during purification: Buffer A: 0.2 M NaOH; Buffer B: 20 mM sodium phosphate, 150 mM NaCl pH 7.2 (Or PBS 1×); Buffer C: 50 mM sodium citrate (dihydrate), pH 3.5; Buffer D: 50 mM succinic acid; and Buffer E: 20% ethanol. The antibodies were purified using the following protocol: 1) the mAbSelect SuRe column was sanitized with 6 column volumes (CV) Buffer A for 15 minutes of contact time (2 mL/min for 15 minutes); 2) the column was rinsed with 5 CV MilliQ water; 3) the column was equilibrated with 15 CV Buffer B. Prior to loading the antibody, 0.5 mL conditioned media was collected and stored; 4) the antibody was loaded onto the column; 5) the column was washed with 6 CV Buffer B; 6) the antibody was eluted using 6 CV Buffer C and the eluate was neutralized with 1/10 volume 600 mM sodium phosphate dibasic or Tris pH 10; 7) the column was stripped using 6 CV Buffer D, and 1/10 volume 600 mM Sodium phosphate dibasic or Tris pH 10 was added to neutralize; 8) the column was cleaned using 10 CV Buffer A; 9) the column was rinsed with 5 CV MilliQ water; 10) the column was re-equilibrated with 10 CV Buffer V; and 11) the column was washed with 10 CV Buffer E and stored at 4° C.

The eluate was reduced to a volume of about 0.5 mL to 2 mL using a MWCO Millipore concentrator: a) about 2 mL of DPBS was added to the concentrator to wet the membrane, the concentrator was centrifuged for about 1 minute, and the liquid was discarded; b) the elute was added to the concentrator and was centrifuged to pass the liquid through the membrane. This was repeated to reduce the volume to about 0.5 mL to 2 mL.

Buffer exchange into DPBS was performed using a GE PD-10 column or a THERMO SCIENTIFIC™ ZEBA™ column: a) the column was cut diagonally to allow for better drippage, and the cap was removed to drain the liquid; b) DPBS (5 mL) was added to and run through the column, and this was repeated for a total of 4 times; c) the entire concentrated sample was loaded by filter sterilizing into the column and was flowed through. The bottom of the MWCO filter was rinsed to collect any extra antibody; d) DPBS was added to bring the volume up to 2 mL to fully load the column; e) a 15 mL conical tube was placed under the column, and 4 mL DPBS was added to the column to elute. The eluate was concentrated using the MWCO Millipore concentrator, and the concentration of the antibody was determined using a NANODROP™ UV-Vis Spectrophotometer.

### Example 3. Cross Reactivity of Clone CA10 Variants to Human and Cyno BCMA Proteins

A solution of BCMA antigen (2 μg/mL or 1 mg/mL biotinylated BCMA antigen when Streptavidin coated plate is used) was prepared in phosphate buffered saline (PBS). The antigen solution was added to 96-well plates (50 μL per well) and was incubated at 4° C. overnight. The plates were blocked with 250 μL per well of PBSA (PBS containing 1% BSA) for 2 hr at 37° C., then washed with 200 μL per well of phosphate buffered saline (PBS) three times. Hybridoma sup (50 μL) or 1 mg/mL of purified test or control (positive and negative) antibody (50 μL) were added per well in the 96-well plate and were incubated for 1 hour at room temperature. The plates were washed with 200 μl per well of PBS, three times, then horse radish peroxidase (HRP) conjugated secondary antibody diluted at 1:5,000 in PBSA (50 μl per well) was added and incubated for 1 hour at room temperature. Then, the plates were washed with 200 μl per well of PBS, three times. The tetramethylbenzidine dihydrochloride (TMB) substrate was prepared by mixing with both Solution A and Solution B (1:1) in a 15 mL conical tube, then 50 μL of the substrate was added to the wells. The color was allowed to develop for 1-5 minutes, then 50 μL of the stop solution was added to each well. The absorbance was read using a SpectraMax (Molecular Devices) at 450 nm.

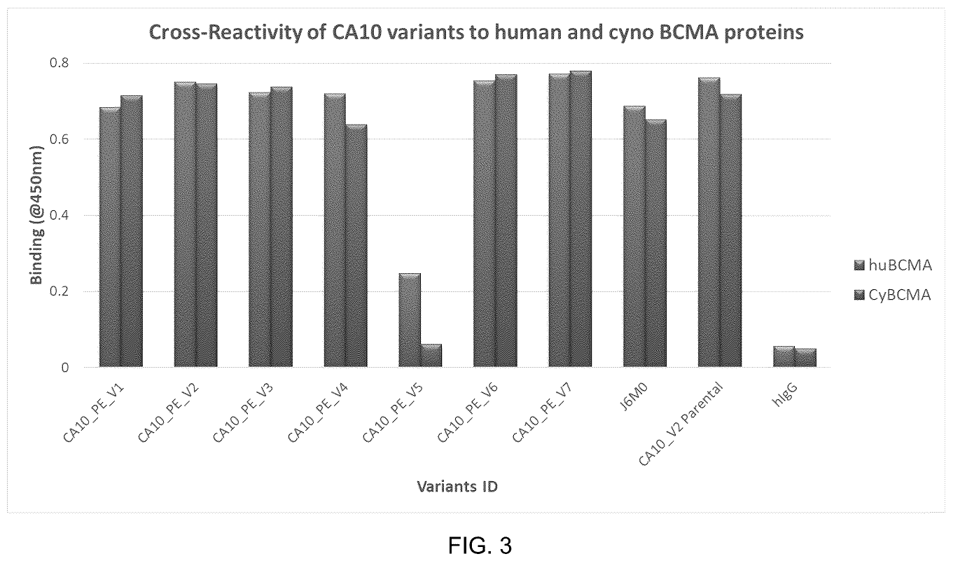

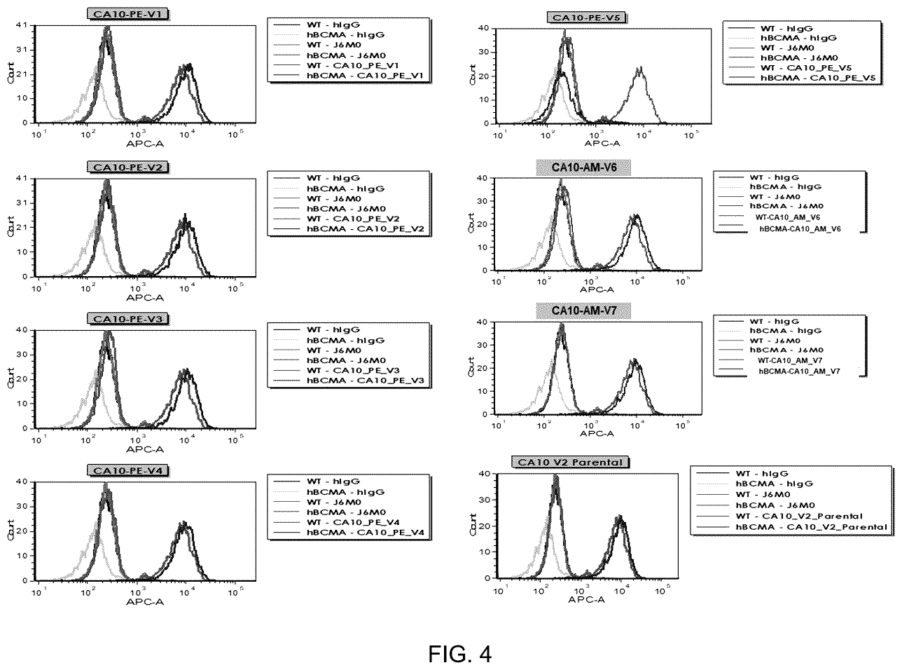

Among the antibody clones that exhibited BCMA specific binding, clone CA10_parental (CA10_V2_parental) showed strong binding (FIG. 3). Variants 1-4, Variant 6, and Variant 7 all had similar binding as the parental antibody. However, variant 5 (CA10_PE_V5) lost binding compared to the other variants (FIG. 3).

### Example 4. Binding Between Anti-BCMA mAb Antibodies and BCMA Proteins on HEK or MM1R Cell Membrane Surfaces

### Cell Staining for Flow Cytometry

HEK 293 cells or MM1R cells (0.5×10⁶) were aliquoted into separate FACS tubes and were labelled as unstained, test sample or positive control. Hybridoma sup (20 μL) or purified CA10 or positive control (1 μg/mL, 5 μL) were added to the cells and incubated on ice for 30 minutes. Cells were washed by adding 3 mL of 1XPBX/5% FBS (FACS Buffer) and were centrifuged for 5-7 minutes at 1500 rpm (450× g). The supernatant was poured off and the cells were gently vortexed to mix. 50 μL of 1:500 dilution of FITC conjugated goat anti-human (or mouse) H+L specific secondary antibody was added to the cells, and the cells were incubated on ice or at 4° C. for 30 minutes in the dark. FACS buffer (3 mL) was added, and cells were washed at 1500 rpm for 5-7 minutes. The supernatant was discarded, and the cells were resuspended in 150-200 μL of FACS buffer. The cells were acquired on a BD FACS Canto and data analyzed using Flojo V10. Software.

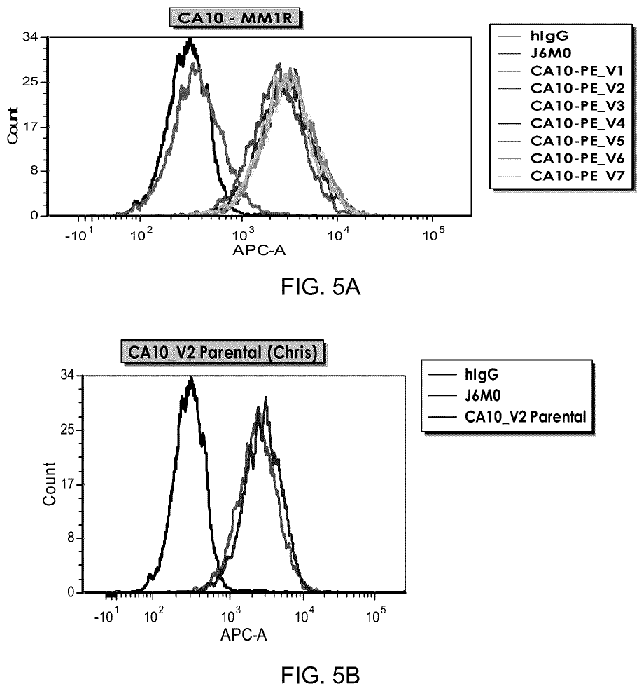

Variants 1-4, Variant 6, and Variant 7 all had similar binding to BCMA protein as the CA10_parental (CA10_V2_parental) antibody on both HEK293 cells and MM1R cells (FIGS. 4 and 5A-5B). Variant 5 showed no binding to BCMA protein on HEK293 cells and MM1R cells.

### Example 5. Antibody and Antigen Protein Interactions and Affinity Constants Determined by Surface Plasmon Resonance Measurements

1×HBS-EP+ was prepared in MilliQ water, filtered, and degassed. A BIACORE™ T100 was primed with 1×HBS-EP+. The immobilized chip Protein A CM % BIACORE™ control software was used.

The antibodies were diluted to 20 μg/mL in HBS-EP+. Seven 1:1 serial dilutions of the antigens were prepared (80 nM final concentration), and 1×HBS-EP+ was used as a 0 nm control. 1×HBS-EP+ was used for start-up injections and HCl (12 mM) was used for regeneration. The samples were run, and the data were analyzed using the BIACORE™ T100 software.

TABLE 3
List of reagents and equipment

| Item | Vendor | Catalog#/Lot |
|---|---|---|
| 10X HBS-EP+ | GE Life sciences | BR-1006-69 |
| 1M HCl | Sigma Aldrich | H1758 |
| 7 mm tubes | GE Life sciences | BR-1002-12 |
| Orange rubber caps type3 | GE Life sciences | BR-1005-02 |
| 15 mm tubes | GE Life sciences | BR-1006-54 |
| Orange rubber caps type5 | GE Life sciences | BR-1006-55 |

TABLE 4
List of antigens

| | | Conc. | | Dilution |
|---|---|---|---|---|
| | | (mg/mL) | | (80 nM |
| | | Make 1/10 | | final). |
| | PPB | stock | Mw (da) | Make 2 mL |
| Human DTA His | 6031 | 2.15 | 28,097 | 20 |
| Cyno DTA His | 17990 | 5.55 | 28,203 | 2 |
| Human BCMA-Avi-His | 5176 | 1.8 | 8,402 | 7.4 |
| Cyno BCMA-Avi-His | 6391 | 1.0 | 8,508 | 6.8 |

### Calculations Used for Preparing the Antigen Dilutions:

$$\frac{Concentration ( \frac{mg}{mL} )}{MW ( kda} Molarity ( M ) , multiply by 1 , 000 , 000 to convert to nM \frac{target concentration ( nM ) \times ( volume required in μ L )}{molarity ( nM )} = μ L of antigen to add$$

TABLE 5
List of antibodies

| | | | Dilution |
|---|---|---|---|
| | | Conc. | (5 ug/mL) 1000 uL |
| | PPB | (mg/mL) | (make 2 × 1 mL) |
| aBCMA A1 | Tara | 3.45 | 1.4 |
| aBCMA B12 | Tara | 1.14 | 4.4 |
| aBCMA CA10 v1 | 21959 | 3.27 | 1.5 |
| aBCMA CA10 v2 | 21960 | 3.32 | 1.5 |
| aBCMA CA17 | 21955 | 3.37 | 1.5 |
| J6M0 hIgG | 1388 | 5.27 | 0.95 |
| BB2121 | 13109 | 1.34 | 3.7 |
| Isotype control: | 5359 | 1.95 | 2.6 |
| Pog IgG1 | | | |

### Calculations Used for Preparing the Antibody Dilutions:

$$\frac{20 μ g / mL}{Antibody concentration ( \frac{mg}{mL} )} = μ L antibody ( based on 1 mL total volume preparation )$$

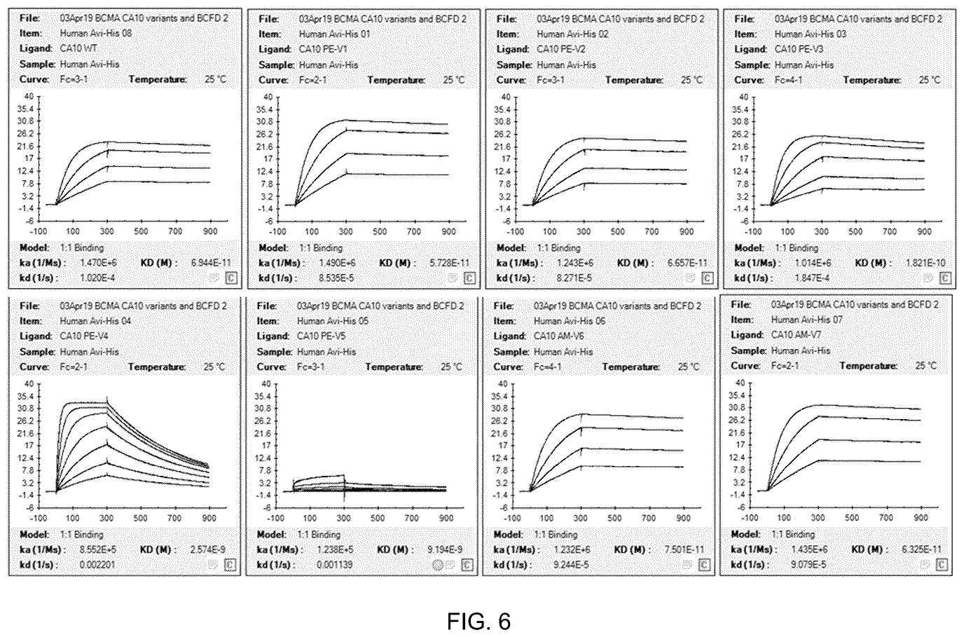

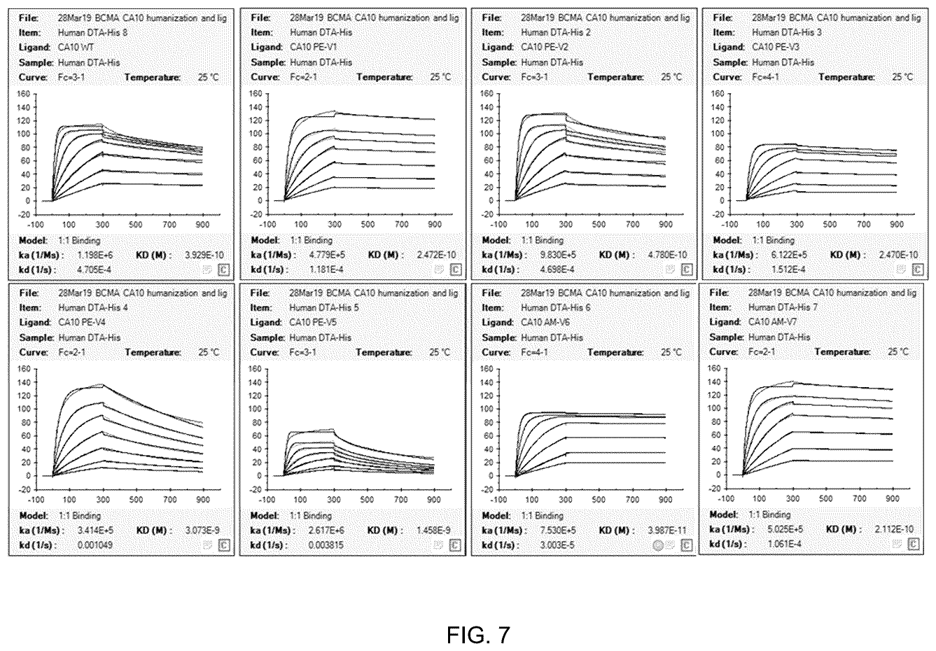

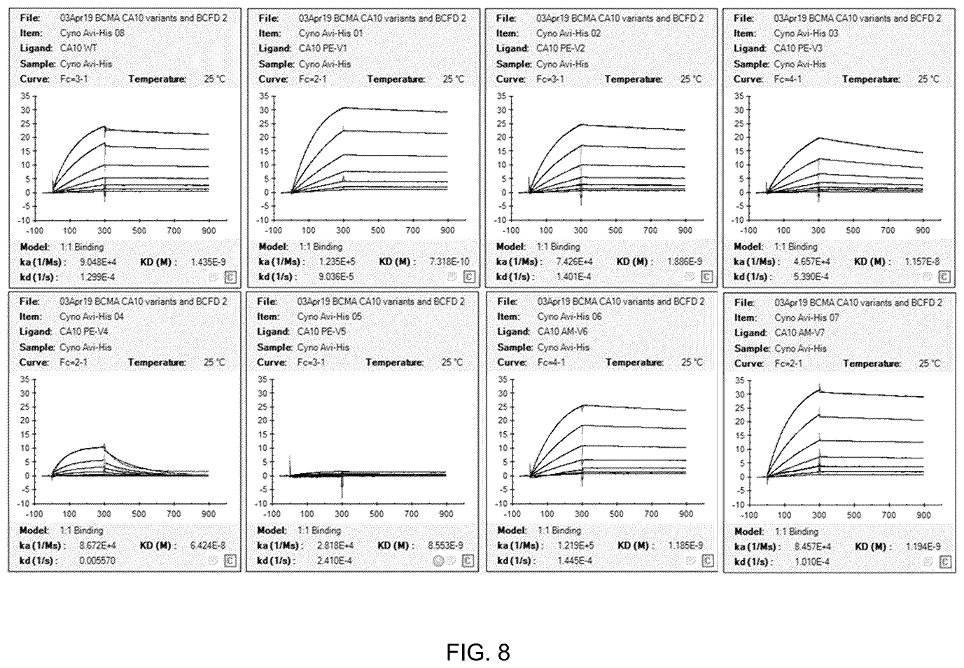

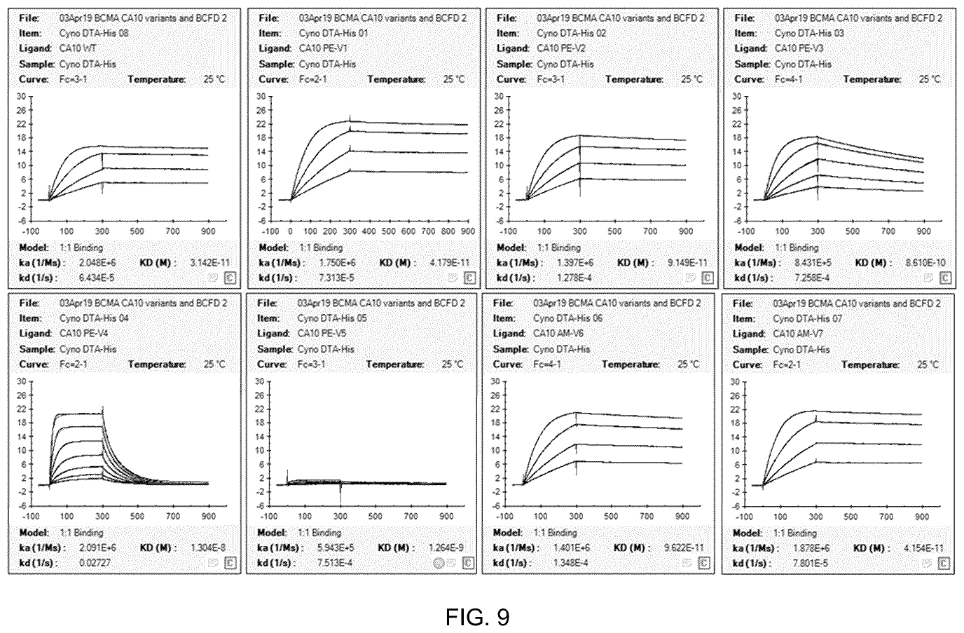

Variants 1, 2, 6, and 7 showed similar affinity as the CA10_parental (CA10 WT) antibody to both human and cyno BCMA proteins (FIG. 6-FIG. 9). Variant 3 showed similar affinity as the parental antibody to the human BCMA protein, but had lower affinity to cyno BCMA. Variants 4 and 5 both lost affinity to human and cyno BCMA proteins.

1. An antibody or antigen-binding fragment thereof that specifically binds to B-cell maturation antigen (BCMA), comprising an antibody heavy chain variable (VH) domain and an antibody light chain variable (VL) domain, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence of CX¹SSTGX²VTPX³X⁴YAN (SEQ ID NO: 25), wherein X¹ is R or A, X² is T or A, X³ is S or G, and X⁴ is N or Y;
a CDR-L2 sequence comprising the amino acid sequence of DNNX⁵X⁶PP (SEQ ID NO: 26),
wherein X⁵ is S, I, or N and X⁶ is R or K; and
a CDR-L3 sequence comprising the amino acid sequence of ALX⁷X⁸GX⁹QWV (SEQ ID NO: 27), wherein X⁷ is W or Y, X⁸ is F or Y, and X⁹ is N or G.

2. The antibody or antigen-binding fragment thereof of claim 1, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence of CASSTGTVTPSNYAN (SEQ ID NO: 4), CRSSTGTVTPSNYAN (SEQ ID NO: 5), CASSTGAVTPSNYAN (SEQ ID NO: 6), or CASSTGAVTPGYYAN (SEQ ID NO: 7);
a CDR-L2 sequence comprising the amino acid sequence of DNNSRPP (SEQ ID NO: 9), DNNIKPP (SEQ ID NO: 10), or DNNNKPP (SEQ ID NO: 11); and
a CDR-L3 sequence comprising the amino acid sequence of ALWFGNQWV (SEQ ID NO: 13), ALWYGGQWV (SEQ ID NO: 14), or ALYYGGQWV (SEQ ID NO: 15).

3. The antibody or antigen-binding fragment thereof of claim 1, wherein:
the VH domain is at least about 90% identical or at least 95% identical to the amino acid sequence of SEQ ID NO: 16; and
the VL domain is at least about 90% identical or at least 95% identical to the amino acid sequence of SEQ ID NO: 17, SEQ ID NO: 18, SEQ ID NO: 19, SEQ ID NO: 20, SEQ ID NO: 21, SEQ ID NO: 23, or SEQ ID NO: 24,
optionally wherein:
the VH domain comprises the amino acid sequence of SEQ ID NO: 16; and
the VL domain comprises an amino acid sequence selected from the group consisting of SEQ ID NO: 17, SEQ ID NO: 18, SEQ ID NO: 19, SEQ ID NO: 20, SEQ ID NO: 21, SEQ ID NO: 23, and SEQ ID NO: 24.

4. The antibody or antigen-binding fragment thereof of claim 3, wherein: the antibody heavy chain comprises the amino acid sequence of SEQ ID NO: 16; and
the antibody light chain comprises the amino acid sequence of SEQ ID NO: 24.

5. The antibody or antigen-binding fragment thereof of claim 1, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence of CASSTGTVTPSNYAN (SEQ ID NO: 4);
a CDR-L2 sequence comprising the amino acid sequence of DNNSRPP (SEQ ID NO: 9); and
a CDR-L3 sequence comprising the amino acid sequence of ALWFGNQWV (SEQ ID NO: 13).

6. The antibody or antigen-binding fragment thereof of claim 5, wherein:
the antibody heavy chain is at least about 90% identical or at least 95% identical to the amino acid sequence of SEQ ID NO: 16; and
the antibody light chain is at least about 90% identical or at least 95% identical to the amino acid sequence of SEQ ID NO: 18,
optionally wherein:
the antibody heavy chain comprises the amino acid sequence of SEQ ID NO: 16; and
the antibody light chain comprises the amino acid sequence of SEQ ID NO: 18.

7. The antibody or antigen-binding fragment thereof of claim 1, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence of CRSSTGTVTPSNYAN (SEQ ID NO: 5);
a CDR-L2 sequence comprising the amino acid sequence of DNNSRPP (SEQ ID NO: 9); and
a CDR-L3 sequence comprising the amino acid sequence of ALWFGNQWV (SEQ ID NO: 13).

8. The antibody or antigen-binding fragment thereof of claim 7, wherein:
the antibody heavy chain is at least about 90% identical or at least 95% identical to the amino acid sequence of SEQ ID NO: 16; and
the antibody light chain is at least about 90% identical or at least 95% identical to the amino acid sequence of SEQ ID NO: 19.

9. The antibody or antigen-binding fragment thereof of claim 1, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence of CASSTGTVTPSNYAN (SEQ ID NO: 4);
a CDR-L2 sequence comprising the amino acid sequence of DNNSRPP (SEQ ID NO: 9); and
a CDR-L3 sequence comprising the amino acid sequence of ALWFGNQWV (SEQ ID NO: 13).

10. The antibody or antigen-binding fragment thereof of claim 9, wherein:
the antibody heavy chain is at least about 90% identical or at least 95% identical to the amino acid sequence of SEQ ID NO: 16; and
the antibody light chain is at least about 90% identical or at least 95% identical to the amino acid sequence of SEQ ID NO: 20.

11. The antibody or antigen-binding fragment thereof of claim 1, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence of CASSTGAVTPSNYAN (SEQ ID NO: 6);
a CDR-L2 sequence comprising the amino acid sequence of DNNIKPP (SEQ ID NO: 10); and
a CDR-L3 sequence comprising the amino acid sequence of ALWYGGQWV (SEQ ID NO: 14).

12. The antibody or antigen-binding fragment thereof of claim 11, wherein:
the antibody heavy chain is at least about 90% identical or at least 95% identical to the amino acid sequence of SEQ ID NO: 16; and
the antibody light chain is at least about 90% identical or at least 95% identical to the amino acid sequence of SEQ ID NO: 21.

13. The antibody or antigen-binding fragment thereof of claim 1, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence CASSTGAVTPGYYAN (SEQ ID NO: 7);
a CDR-L2 sequence comprising the amino acid sequence of DNNNKPP (SEQ ID NO: 11); and
a CDR-L3 sequence comprising the amino acid sequence of ALYYGGQWV (SEQ ID NO: 15).

14. The antibody or antigen-binding fragment thereof of claim 13, wherein:
the antibody heavy chain is at least about 90% identical or at least 95% identical to the amino acid sequence of SEQ ID NO: 16; and
the antibody light chain is at least about 90% identical or at least 95% identical to the amino acid sequence of SEQ ID NO: 23.

15. The antibody or antigen-binding fragment thereof of claim 1, wherein:
the VH domain comprises
a CDR-H1 sequence comprising the amino acid sequence of GFTFSNFGMH (SEQ ID NO: 1);
a CDR-H2 sequence comprising the amino acid sequence of VIWSDETNR (SEQ ID NO: 2); and
a CDR-H3 sequence comprising the amino acid sequence of DQQYCSSDSCFTWFDP (SEQ ID NO: 3); and
the VL domain comprises
a CDR-L1 sequence comprising the amino acid sequence of CASSTGTVTPSNYAN (SEQ ID NO: 4);
a CDR-L2 sequence comprising the amino acid sequence of DNNSRPP (SEQ ID NO: 9); and
a CDR-L3 sequence comprising the amino acid sequence of ALWFGNQWV (SEQ ID NO: 13).

16. The antibody or antigen-binding fragment thereof of claim 1, wherein:
the antibody or antigen-binding fragment thereof is a chimeric or humanized antibody or antigen-binding fragment thereof, or
the antibody or antigen-binding fragment thereof is a human antibody or antigen-binding fragment thereof.

17. The antibody or antigen-binding fragment thereof of claim 1, wherein the antibody or antigen-binding fragment thereof is a multi-specific antibody.

18-22. (canceled)

23. A pharmaceutical composition comprising the antibody or antigen-binding fragment thereof according to claim 1.

24. An isolated nucleic acid molecule comprising a nucleotide sequence that encodes the binding protein according to claim 1.

25. An expression vector comprising the nucleic acid molecule of claim 24.

26. A host cell comprising the nucleic acid molecule of claim 24.

27. A host cell comprising the expression vector of claim 25.

28. (canceled)

29. A method for making an antibody or antigen-binding fragment thereof, comprising culturing the host cell of claim 26 under suitable conditions and recovering the antibody or antigen-binding fragment thereof.

30. A method of treating or preventing a disease or disorder, the method comprising administering to a subject in need thereof the pharmaceutical composition of claim 23.

31. A method of treating or preventing a cancer, the method comprising administering to the subject in need thereof the pharmaceutical composition of claim 23.

32. A method of treating or preventing multiple myeloma, the method comprising administering to the subject in need thereof the pharmaceutical composition of claim 23.

33. A method of treating or preventing a plasma cell disorder, the method comprising administering to the subject in need thereof the pharmaceutical composition of claim 23.
